(19) United States
(12) Patent Application Publication
(10) Pub. No.: US 2005/0014783 A1
(43) Pub. Date: Jan. 20, 2005

(54) Use of Rho-kinase inhibitors in the treatment of aneurysm and cardiac hypertrophy

(75) Inventors: William P. Dole, Alamo, CA (US); David Light, San Mateo, CA (US); Baby Martin-McNulty, Mill Valley, CA (US); Sabine Schirm, El Cerrito, CA (US); Yi-Xin Wang, Lafayette, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/857,062

(22) Filed: May 28, 2004

Related U.S. Application Data
(60) Provisional application No. 60/474,141, filed on May 29, 2003.

(51) Int. Cl. A61K 31/47

(52) U.S. Cl. 514/310

(57) ABSTRACT

The present invention is directed to a method for preventing or decreasing the incidence of or treating aneurysm or cardiac hypertrophy, comprising administering an effective amount of a rho-kinase inhibitor, such as fasudil, to a subject in need thereof.

This application claims priority to U.S. Provisional Patent Application Ser. No. 60/474,141 filed May 29, 2003 the entirety of which is incorporated herein by reference.

### FIELD OF THE INVENTION

This invention relates to the use of Rho-kinase inhibitors, such as isoquinoline compounds (e.g. fasudil), in the treatment and/or prevention of aneurysm and cardiac hypertrophy.

### BACKGROUND OF THE INVENTION

The successful treatment of certain cardiovascular diseases has been hampered by the lack of effective medicaments for their treatment. These diseases include abdominal aortic aneurysm and cardiac hypertrophy. Abdominal aortic aneurysm, which is often a result of atherosclerosis, is a bulge in the aorta, the main blood vessel coming from the heart that supplies blood to all organs, and can prove fatal if left untreated. In addition to the proteolytic mechanism described in Wang (2001) Am J Pathology, 159:1455-1464, apoptosis is also involved in aneurysm formation (Henderson (1999) Circulation, 99:96-104). Cardiac hypertrophy, which is an adaptive response to hemodynamic or non-hemodynamic stimuli, may occur as the result of a variety of ailments including, but not limited to, high blood pressure, valvular heart disease, myocardial infarction, and cardiomyopathy, and leads to an enlarged heart.

Rho-kinase has been identified as one of the effectors of the small GTP-binding protein Rho. Recently, the Rho/Rho-kinase mediated pathway has been the subject of much investigation (H. Shimokawa (2002) J. Cardiovascular Pharm. 39: 319-327). Rho-kinase inhibitors have been disclosed as being advantageously used as vasodilators, cerebral circulation ameliorators, and antihypertensive agents.

Clinical experience, however, does not suggest that antihypertensive agents alone are an effective treatment of abdominal aortic aneurysm and calcium channel blockers, which are often given to patients diagnosed with hypertension and which decrease blood pressure, may increase risk in patients with abdominal aortic aneurysm (Wilmink et al. (2002) J. Vasc. Surg. 36:751-757).

### SUMMARY OF THE INVENTION

The present invention involves the novel use of Rho-kinase inhibitors in the treatment of aneurysim (including the related disorders of atherosclerosis and/or stenosis) and cardiac hypertrophy.

Numerous classes of compounds that exhibit Rho-kinase inhibitory activity are known in the art, including isoquinoline compounds of the following formula I: 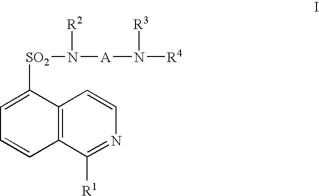
including salts and solvates thereof wherein
R¹ is H, Cl, or OH;
   When R¹ is H,
      A is an ethylene group optionally substituted with C₁₋₆ alkyl, phenyl or benzyl;
      R² and R³ are directly bonded to each other thereby forming a trimethylene group optionally substituted with C₁₋₆ alkyl, phenyl or benzyl;
      R⁴ is H or C₁₋₆ alkyl; and
   When R¹ is Cl or OH
      A is a C₂₋₆ alkylene group optionally substituted with C₁₋₆ alkyl;
      R² and R³ are not bonded to each other and each is independently H or C₁₋₆ alkyl, or R² and R³ are directly bonded with each other thereby forming either an ethylene group optionally substituted with C₁₋₆ alkyl, or a trimethylene group optionally substituted with C₁₋₆ alkyl and
      R⁴ is H, C₁₋₆ alkyl or amidino.

Compounds of formula I are disclosed in U.S. Pat. No. 4,678,783, the entirety of which is incorporated herein by reference.

Compounds of formula I include the compound fasudil (CAS 103745-39-7, HA-1077, AT-877), which has been shown to be an inhibitor of Rho-kinase activity (Asano et al. (1987) J. Pharmacol. Exp. Ther. 24:1033-1040). Fasudil, (or Hexahydro-1-(5-isoquinolylsulfonyl)-1H-1,4-diazepine) has the structure of formula II:

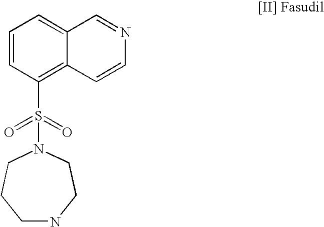

Fasudil has been described as the therapeutic drug of choice in treating cerebral vasospasm subsequent to subarachnoid hemorrhage (see e.g., U.S. Pat. No. 6,153,608), and has been suggested for use in treatment of ischemic coronary syndrome caused by coronary artery spasm (Matsumoto et al. (2002) Circulation 105:1545-1547).

It has recently been demonstrated that hydroxyfasudil (III), a major active metabolite of fasudil, has a more specific inhibitory effect on Rho-kinase (Shimokawa et al. (1999) Cardiovas. Res. 43:1138-1141).

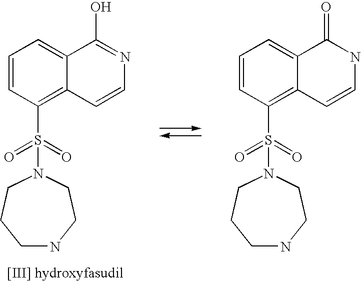

Known Rho-kinase inhibitors further include the compound Y-27632 (IV) and Wf-536 (V): 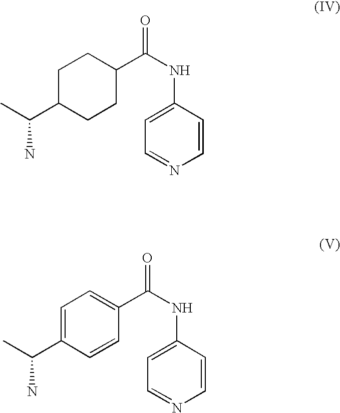
and similar compounds disclosed in U.S. Pat. No. 4,997,834, U.S. Pat. No. 6,451,825, WO 95/28387, WO 00/078,351, WO 00/057,913, EP 1295607, and EP 00187371, of which the entirety of each is incorporated herein by reference.

Known Rho-kinase inhibitors further include compound (VI) and compounds disclosed in U.S. 2003/0087919.:

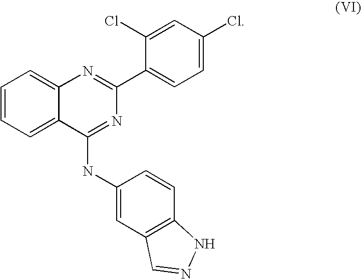

Toward this end, the present invention provides a method for the treatment and/or prevention of aneurysm (especially abdominal aortic aneurysm) and cardiac hypertrophy, in a subject in need thereof, comprising administering a therapeutically effective amount of a Rho-kinase inhibitor.

In a preferred embodiment, the Rho-kinase inhibitor is an isoquinoline derivative (especially an isoquinoline derivative of formula I).

In a further embodiment, the isoquinoline Rho-kinase inhibitor is fasudil, hydroxyfasudil or a pro-drug ester thereof.

In another aspect, the invention provides pharmaceutical compositions comprising a Rho-kinase inhibitor and a pharmaceutically acceptable excipient.

In another aspect, the invention relates to administration of a Rho-kinase inhibitor, to a subject in need thereof, in combination with other agents or drugs used in the treatment of cardiovascular ailments, selected from the group consisting of cholesterol lowering agents, antihypertensive agents, beta blocker drugs, calcium channel blockers, diuretics, nitrates, and ACE inhibitors.

### DESCRIPTION OF THE FIGURES

FIG. 1: Effect of fasudil treatment on aneurysm formation. Apolipoprotein E deficient (ApoE-KO) mice were treated with angiotensin II (see Example 1), in the absence or presence of fasudil at concentrations of 0.5 mg/ml and 1 mg/ml in drinking water. C57 Black mice is the mouse strain from which the ApoE-KO mouse is derived. Statistically significant differences are indicated as follows: * p<0.05, versus Control (ApoE-KO without Ang II treatment), and # p<0.05 versus Ang II treated ApoE-KO mice. Fasudil treatment led to a 29% decrease in the size of aneurysms found in Ang II treated ApoE-KO mice.

FIG. 2: Effects of fasudil treatment on apoptosis detected by TUNEL. H & E staining reveals that the thickening of the abdominal aortic wall caused by Ang II treatment is associated with adventitial fibrosis, inflammation, and destruction of smooth muscle cells and elastin in the vascular media. These changes are ameliorated, but not fully eliminated, by high dose fasudil treatment in animals with aneurysm (top). Ang II treatment is also seen to cause widespread apoptosis in both the media and adventitia of the supra-renal aortic wall in apoE-KO mice. Fasudil has the effect of reducing apoptosis (especially in the vessel media) as shown by TUNEL staining.

FIG. 3. To further quantify apoptosis in the aortic tissue, cell death detection ELISA assay was performed. Consistent with TUNEL assay, cytosolic DNA fragments were significantly increased in Ang II-treated apoE-KO mice compared to that in vehicle controls, p<0.01. The increased cytosolic DNA fragmentation by Ang II was abolished by fasudil treatment, P<0.01.

FIG. 4: Effect of fasudil treatment on the degree of cardiac hypertrophy. ApoE-KO mice were treated with angiotensin II (see Example 1), in the absence and presence of fausdil (at concentrations of 0.5 mg/ml and 1 mg/ml). The C57 Black mice is the mouse strain from which the ApoE-KO is derived. Statistically significant differences are indicated as follows: * p<0.05, versus Control (ApoE-KO without Ang II treatment), and # p<0.05 versus Ang II treated ApoE-KO mice. Fasudil treatment led to a 14% decrease in the degree of cardiac hypertrophy found in Ang II treated ApoE-KO mice.

### DETAILED DESCRIPTION OF THE INVENTION

Definitions

As used in the specification and appended claims, unless specified to the contrary, the following terms have the meaning indicated:

The term “alkyl” refers to straight or branched chain unsubstituted hydrocarbon groups of 1 to 20 carbon atoms, preferably 1 to 7 carbon atoms. The expression “lower alkyl” refers to unsubstituted alkyl groups of 1 to 4 carbon atoms. When a subscript is used with reference to an alkyl or other group, the subscript refers to the number of carbon atoms that the group may contain. For example, the term “C₀₋₄alkyl” includes a bond and alkyl groups of 1 to 4 carbon atoms.

The term “substituted alkyl” refers to an alkyl group substituted by one to four substituents selected from halogen, hydroxy, alkoxy, keto (═O), alkanoyl, aryloxy, alkanoyloxy, NRₐR_{b}, alkanoylamino, aroylamino, aralkanoylamino, substituted alkanoylamino, substituted arylamino, substituted aralkanoylamino, thiol, alkylthio, arylthio, aralkylthio, alkylthiono, arylthiono, aralkylthiono, alkylsulfonyl, arylsulfonyl, aralkylsulfonyl, —SO₂NRₐR_{b}, nitro, cyano, —CO₂H, —CONRₐR_{b}, alkoxycarbonyl, aryl, guanidino and heteroaryls or heterocyclos (such as indolyi, imidazolyl, furyl, thienyl, thiazolyl, pyrrolidyl, pyridyl, pyrimidyl and the like), wherein Rₐ and R_{b} are selected from hydrogen, alkyl, aryl, aralkyl, cycloalkyl, cycloalkylalkyl, heteroaryl, heteroarylalkyl, heterocycle, and heterocyclealkyl. The substituent on the alkyl optionally in turn may be further substituted, in which case it will be with substituted one or more of C₁₋₄alkyl, C₂₋₄alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenyloxy, and/or benzyloxy.

The term “alkenyl” refers to straight or branched chain hydrocarbon groups of 2 to 20 carbon atoms, preferably 2 to 15 carbon atoms, and most preferably 2 to 8 carbon atoms, having at least one double bond, and depending on the number of carbon atoms, up to four double bonds.

The term “substituted alkenyl” refers to an alkenyl group substituted by one to two substituents selected from those recited above for substituted alkyl groups.

The term “alkynyl” refers to straight or branched chain hydrocarbon groups of 2 to 20 carbon atoms, preferably 2 to 15 carbon atoms, and most preferably 2 to 8 carbon atoms, having at least one triple bond, and depending on the number of carbon atoms, up to four triple bonds.

The term “substituted alkynyl” refers to an alkynyl group substituted by one to two substituents selected from those recited above for alkyl groups.

When the term alkyl is used in connection with another group, as in heterocycloalkyl or cycloalkylalkyl, this means the identified (first named) group is bonded directly through an alkyl group which may be branched or straight chain (e.g., cyclopropylC₁₋₄alkyl means a cyclopropyl group bonded through a straight or branched chain alkyl group having one to four carbon atoms.). In the case of substituents, as in “substituted cycloalkylalkyl,” the alkyl portion of the group, besides being branched or straight chain, may be substituted as recited above for substituted alkyl groups and/or the first named group (e.g., cycloalkyl) may be substituted as recited herein for that group.

The term “halogen” or “halo” refers to fluorine, chlorine, bromine and iodine.

The term “aryl” refers to monocyclic or bicyclic aromatic substituted or unsubstituted hydrocarbon groups having 6 to 12 carbon atoms in the ring portion, such as phenyl, naphthyl, and biphenyl groups. Each ring of the aryl may be optionally substituted with one to three R_{c} groups, wherein R_{c} at each occurrence is selected from alkyl, substituted alkyl, halogen, trifluoromethoxy, trifluoromethyl, —SR, —OR, —NRR′, —NRSO₂R′, —SO₂R, —SO₂NRR′, —CO₂R′, —C(═O)R′, —C(═O)NRR′, —OC(═O)R′, —OC(═O)NRR′, —NRC(═O)R′, —NRCO₂R′, phenyl, C₃₋₇ cycloalkyl, and five-to-six membered heterocyclo or heteroaryl, wherein each R and R′ is selected from hydrogen, alkyl, substituted alkyl, alkenyl, substituted alkenyl, phenyl, C₃₋₇cycloalkyl, and five-to-six membered heterocyclo or heteroaryl, except in the case of a sulfonyl group, then R is not going to be hydrogen. Each substituent R_{c} optionally in turn may be further substituted by one or more (preferably 0 to 2) R_{d} groups, wherein R_{d} is selected from C₁₋₆alkyl, C₂₋₆alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenylethyl, phenyloxy, and benzyloxy.

The term “aralkyl” refers to an aryl group bonded directly through an alkyl group, such as benzyl, wherein the alkyl group may be branched or straight chain. In the case of a “substituted aralkyl,” the alkyl portion of the group besides being branched or straight chain, may be substituted as recited above for substituted alkyl groups and/or the aryl portion may be substituted as recited herein for aryl.

Thus, the term “optionally substituted benzyl” refers to the group 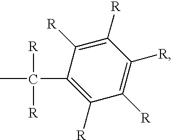
wherein each R group may be hydrogen or may also be selected from R_{c} as defined above, in turn optionally substituted with one or more R_{d}. At least two of these “R” groups should be hydrogen and preferably at least five of the “R” groups is hydrogen. A preferred benzyl group involves the alkyl-portion being branched to define

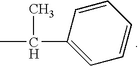

The term “heteroaryl” refers to a substituted or unsubstituted aromatic group for example, which is a 4 to 7 membered monocyclic, 7 to 11 membered bicyclic, or 10 to 15 membered tricyclic ring system, which has at least one heteroatom and at least one carbon atom-containing ring. Each ring of the heteroaryl group containing a heteroatom can contain one or two oxygen or sulfur atoms and/or from one to four nitrogen atoms, provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. It may optionally be substituted with one to three (preferably 0 to 2) R_{c} groups, as defined above for aryl, which in turn may be substituted with one or more (preferably 0 to 2) R_{d} groups, also as recited above.

Exemplary monocyclic heteroaryl groups include pyrrolyl, pyrazolyl, pyrazolinyl, imidazolyl, oxazolyl, isoxazolyl, thiazolyl (i.e. 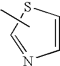
), thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

The term “cycloalkyl” refers to a saturated or partially unsaturated non-aromatic cyclic hydrocarbon ring system, preferably containing 1 to 3 rings and 3 to 7 carbon atoms per ring, which may be substituted or unsubstituted and/or which may be fused with a C₃-C₇ carbocylic ring, a heterocyclic ring, or which may have a bridge of 3 to 4 carbon atoms. The cycloalkyl groups including any available carbon or nitrogen atoms on any fused or bridged rings optionally may have 0 to 3 (preferably 0-2) substituents selected from R_{c} groups, as recited above, and/or from keto (where appropriate) which in turn may be substituted with one to three R_{d} groups, also as recited above. Thus, when it is stated that a carbon-carbon bridge may be optionally substituted, it is meant that the carbon atoms in the bridged ring optionally may be substituted with an R_{c} group, which preferably is selected from C₁₋₄alkyl, C₂₋₄alkenyl, halogen, haloalkyl, haloalkoxy, cyano, amino, C₁₋₄alkylamino, aminoC₁₋₄alkyl, hydroxy, hydroxyC₁₋₄alkyl, and C₁₋₄alkoxy. Exemplary groups include cyclopropyl, cyclobutyl, cyclopentyl, cyclohexyl, cycloheptyl, bicycloheptane, cycloctyl, cyclodecyl, cyclododecyl, and adamantyl.

The terms “heterocycle”, “heterocyclic” and “heterocyclo” each refer to a fully saturated or partially unsaturated nonaromatic cyclic group, which may be substituted or unsubstituted, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen, oxygen, and sulfur atoms, where the nitrogen and sulfur heteroatoms also optionally may be oxidized and the nitrogen heteroatoms also optionally may be quaternized. Preferably two adjacent heteroatoms are not simultaneously selected from oxygen and nitrogen. The heterocyclic group may be attached at any nitrogen or carbon atom. The heterocyclo groups optionally may have 0 to 3 (preferably 0-2) substituents selected from keto (═O), and/or one or more R_{c} groups, as recited above, which in turn may be substituted with one to three R_{d} groups, also as recited above.

Exemplary monocyclic heterocyclic groups include pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, dioxanyl, isothiazolidinyl, thietanyl, thiiranyl, triazinyl, and triazolyl, and the like.

Exemplary bicyclic hetrocyclic groups include 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl] or furo[2,3-b]pyridinyl), dihydroisoindolyi, dihydroquinazolinyl (such as 3,4-dihydro-4-oxo-quinazolinyl), benzisothiazolyl, benzisoxazolyl, benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

Also included are smaller heterocyclos, such as epoxides and aziridines.

Unless otherwise indicated, when reference is made to a specifically-named aryl (e.g., phenyl), cycloalkyl (e.g., cyclohexyl), heterocyclo (e.g., pyrrolidinyl) or heteroaryl (e.g., indolyl), the reference is intended to include rings having 0 to 3, preferably 0-2, substituents selected from those recited above for the the aryl, cycloalkyl, heterocyclo and/or heteroaryl groups, as appropriate. Additionally, when reference is made to a specific heteroaryl or heterocyclo group, the reference is intended to include those systems having the maximum number of non-cumulative double bonds or less than the maximum number of double bonds. Thus, for example, the term “isoquinoline” refers to isoquinoline and tetrahydroisoquinoline.

Additionally, it should be understood that one skilled in the field may make appropriate selections for the substituents for the aryl, cycloalkyl, heterocyclo, and heteroaryl groups to provide stable compounds and compounds useful as pharmaceutically-acceptable compounds and/or intermediate compounds useful in making pharmaceutically-acceptable compounds.

The term “heteroatoms” shall include oxygen, sulfur and nitrogen.

The term “haloalkyl” means an alkyl having one or more halo substituents.

The term “perfluoromethyl” means a methyl group substituted by one, two, or three fluoro atoms, i.e., CH₂F, CHF₂ and CF₃. The term “perfluoroalkyl” means an alkyl group having from one to five fluoro atoms, such as pentafluoroethyl.

The term “haloalkoxy” means an alkoxy group having one or more halo substituents. For example, “haloalkoxy” includes —OCF₃.

The term “carbocyclic” means a saturated or unsaturated monocyclic or bicyclic ring in which all atoms of all rings are carbon. Thus, the term includes cycloalkyl and aryl rings. The carbocyclic ring may be substituted in which case the substituents are selected from those recited above for cycloalkyl and aryl groups.

When the term “unsaturated” is used herein to refer to a ring or group, the ring or group may be fully unsaturated or partially unsaturated.

Definitions for the various other groups that are recited above in connection with substituted alkyl, substituted alkenyl, aryl, cycloalkyl, and so forth, are as follows: alkoxy is —OR^{e}, alkanoyl is —C(═O)R^{e}, aryloxy is —OAr, alkanoyloxy is —OC(═O)R^{e}, amino is —NH₂, alkylamino is —NHR^{e} or —N(R^{e})₂, arylamino is —NHAr or —NR^{e}Ar, aralkylamino is —NH—R^{f}—Ar, alkanoylamino is —NH—C(═O)R^{e}, aroylamino is —NH—C(═O)Ar, aralkanoylamino is —NH—C(═O)R^{f}—Ar, thiol is —SH, alkylthio is —SR^{e}, arylthio is —SAr, aralkylthio is —S—R^{f}—Ar, alkylthiono is —S(═O)R^{e}, arylthiono is —S(═O)Ar, aralkylthiono is —S(═O)R^{f}—Ar, alkylsulfonyl is —SO_{(q)}R^{e}, arylsulfonyl is —SO_{(q)}Ar, arylsulfonylamine is —NHSO_{(q)}Ar, alkylsulfonylamine is —NHSO₂R^{e}, aralkylsulfonyl is —SO_{(q)}R^{f}Ar, sulfonamido is —SO₂NH₂, substituted sulfonamide is —SO₂NHR^{e} or —SO₂N(R^{e})₂, nitro is —NO₂, carboxy is —CO₂H, carbamyl is —CONH₂, substituted carbamyl is —C(═O)NHR^{g} or —C(═O)NR^{g}R^{h}, alkoxycarbonyl is —C(═O)OR^{e}, carboxyalkyl is —R^{f}—CO₂H, sulfonic acid is —SO₃H, arylsulfonylamine is —NHSO_{(q)}Ar, guanidino is 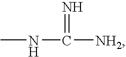
and ureido is 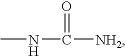
wherein R^{e} is alkyl or substituted alkyl as defined above, R^{f} is alkylene or substituted alkylene as defined above, R^{g} and R^{h} are selected from alkyl, substituted alkyl, aryl, aralkyl, cycloalkyl, heterocyclo, and heteroaryl; Ar is an aryl as defined above, and q is 2 or 3.

“Pharmaceutically acceptable excipient” refers to an acceptable carrier, and any pharmaceutically acceptable auxiliary substance as required to be compatible with physiological conditions, which are non-toxic and do not adversely effect the biological activity of the pharmaceutical composition suspended or included within it. Suitable excipients would be compounds such as mannitol, succinate, glycine, or serum albumin.

“Therapeutically effective amount” refers to that amount of a compound of the invention, which, when administered to a subject in need thereof, is sufficient to effect treatment, as defined below, for patients suffering from, or likely to develop, aneurysm or cardiac hypertrophy. The amount of a compound which constitutes a “therapeutically effective amount” will vary depending on the compound, but can be determined routinely by one of ordinary skill in the art having regard to his own knowledge and to this disclosure.

“Treating” or “treatment” as used herein covers the treatment of aneurysm or cardiac hypertrophy, and includes:
(a) preventing aneurysm or cardiac hypertrophy from occurring in a human, particularly when such human is predisposed to having these conditions;
(b inhibiting aneurysm or cardiac hypertrophy, i.e. arresting its development; or
(c) relieving aneurysm or cardiac hypertrophy, i.e. causing regressing of the conditions.
   Compound Preparation

Compounds of formulae I, IV, V and VI can be prepared as disclosed in publicly available literature.

Fasudil [II] can be obtained from commercial sources (e.g. from Asahi Kasei Corporation of Tokyo, Japan) or it can be synthesized according to conventional methods (U.S. Pat. No. 4,678,783).

Fasudil (hexahydro-1-(5 isoquinolinesulfonyl)-1H-1,4-diazepine) can exist as a hydrochloride salt (C₁₄H₁₇N₃O₂.HCl), with a molecular weight of 327.83. For example, the hydrochloride of compound [II]can be prepared by dissolving compound [II] in sterile water or phosphate buffered saline. A salt of compound [II], according to the invention, includes but is not limited to salts with inorganic acids such as hydrochloric acid, sulfuric acid, nitric acid, phosphoric acid, hydrofluoric acid, and hydrobromic acid, and salts with organic acids such as acetic acid, tartaric acid, lactic acid, citric acid, fumaric acid, maleic acid, succinic acid, methanesulfonic acid, ethanesulfonic acid, benzenesulfonic acid, toluenesulfonic acid, naphthalensulfonic acid, and camphorsulfonic acid. Preferable solvates include hydrates.

The compound of the invention as produced in the above manner can be isolated and purified, in the form of a free base or an acid addition salt, by known procedures. The compound of the invention may show polymorphism, and such compounds all fall within the scope of the invention. It is known to those of ordinary skill in the art that compounds having an isoquinoline ring structure can have various substitutions, e.g. cyclic aminosulfonyl in the 4-position and the 5-position.

Hydroxyfasudil [III], which is produced during in vivo metabolism of fasudil, is among the preferred embodiments of the claimed invention. Also preferred are other derivatives of hydroxy fasudil (i.e. prodrug (VII)), whose metabolism can lead to in vivo production of therapeutic amounts of hydroxyfasudil within the treated subject. 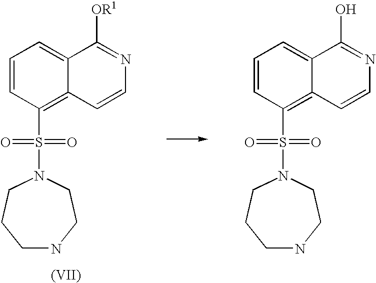
wherein:
R¹ is
   (a) alkyl, cycloalkyl, alkenyl, alkynyl, (cycloalkyl)alkyl, (aryl)alkyl, (heteroaryl)alkyl, (heterocyclo)alkyl, aryl, heteroaryl or heterocyclo any of which may be optionally independently substituted as valence allows with 1 to 3 functional groups; or
   (b) —C(═O)R²;
R² is alkyl, cycloalkyl, alkenyl, alkynyl, (cycloalkyl)alkyl, (aryl)alkyl, (heteroaryl)alkyl, (heterocyclo)alkyl, aryl, heteroaryl or heterocyclo any of which may be optionally independently substituted as valence allows with 1 to 3 functional groups.
   Preferred compounds of formula VII include those wherein R¹ is alkyl.
   Identification of Rho-Kinase Inhibitors

Compounds of the invention can be assayed for activity as a Rho-kinase inhibitor using a kinase activity assay such as that described by Amano et al. (1999) J. Biol. Chem. 274:32418-32424. Compounds are generally considered to be effective inhibitors if they have an IC₅₀ of 10 μM, preferably 5 μM, more preferably 1 μM or less.

Models for Abdominal Aortic Aneurysm and/or Cardiac Hypertrophy

Infusion of apoE-KO mice (Jackson Laboratories, Bar Harbor, Me.) with angiotensin II has been reported to lead to the formation of large abdominal aortic aneurysms (Daugherty et al. (2000) J. Clin. Invest. 105:1605-1612). This system can be used as a model system in which to study the effects of the Rho-kinase inhibitors of the present invention on these conditions, using the methods described in Wang et al. (2001) American J. Path. 159:1455-1464. (See Example 1).

Angiotensin II-induced cardiac hypertrophy has been reported in rats (Kagiyama et al. (2002) Circulation 106(8):909-12) and we have found that the methods used above also produce this disorder in apoE-KO mice.

Pharmaceutical Compositions and Administration of the Compounds of the Invention

Rho-kinase inhibitors of the invention, such as compounds of formulae I, II, III, IV, V, VI and VII can be administered to a patient following conventional procedures, using conventional regimens of administration, kits, modes of administration, and dosages, all of which are well known to those of skill in the art.

Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally, topically, transdermally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages. The compositions will include a conventional pharmaceutical carrier or excipient and a compound of the invention as the/an active agent, and, in addition, may include other medicinal agents, pharmaceutical agents, carriers, adjuvants, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient.

Preferably, the composition will be about 5% to 75% by weight of a compound(s) of the invention, or a pharmaceutically acceptable salt thereof, with the rest being suitable pharmaceutical excipients.

The preferred route of administration is oral, using a convenient daily dosage regimen which can be adjusted according to the degree of severity of the disease-state to be treated. For such oral administration, a pharmaceutically acceptable composition containing a compound(s) of the invention, or a pharmaceutically acceptable salt thereof, is formed by the incorporation of any of the normally employed excipients, such as, for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, and the like. Such compositions take the form of solutions, suspensions, tablets, pills, capsules, powders, sustained release formulations and the like.

Preferably such compositions will take the form of capsule, caplet or tablet and therefore will also contain a diluent such as lactose, sucrose, dicalcium phosphate, and the like; a disintegrant such as croscarmellose sodium or derivatives thereof; a lubricant such as magnesium stearate and the like; and a binder such as a starch, gum acacia, polyvinylpyrrolidone, gelatin, cellulose ether derivatives, and the like.

The compounds of the invention, or their pharmaceutically acceptable salts, may also be formulated into a suppository using, for example, about 0.5% to about 50% active ingredient disposed in a carrier that slowly dissolves within the body, e.g., polyoxyethylene glycols and polyethylene glycols (PEG), e.g., PEG 1000 (96%) and PEG 4000 (4%).

Liquid pharmaceutically administrable compositions can, for example, be prepared by dissolving, dispersing, etc., a compound(s) of the invention (about 0.5% to about 20%), or a pharmaceutically acceptable salt thereof, and optional pharmaceutical adjuvants in a carrier, such as, for example, water, saline, aqueous dextrose, glycerol, ethanol and the like, to thereby form a solution or suspension.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state characterized by abdominal aortic aneurysm or cardiac hypertrophy, in accordance with the teachings of this invention.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157). For example, a solution or suspension of an isoquinoline compound in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of an aryl urea compound may be formulated into a lotion or salve.

Suitable solvents for processing transdermal delivery systems are known to those skilled in the art, and include dimethylsulfoxide, lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery systems are known to those skilled in the art, and include, for example, monohydroxy or polyhydroxy alcohols such as ethanol, propylene glycol or benzyl alcohol, saturated or unsaturated C₈-C₁₈ fatty alcohols such as lauryl alcohol or cetyl alcohol, saturated or unsaturated C₈-C₁₈ fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C₈-C₁₈ fatty alcohols, saturated or unsaturated C₈-C₁₈ fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

It will be appreciated by those skilled in the art that the particular method of administration will depend on a variety of factors, all of which are routinely considered when administering therapeutics. It will also be understood, however, that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of the isoquinoline derivative compound or a pharmaceutically acceptable salt thereof given for a defined number of days, can be ascertained by those skilled in the art using conventional treatment tests.

The Rho-kinase inhibitor of the invention (e.g. compounds of formula I, II) can be administered to a patient at a dosage that can range from about 0.1 to about 300 mg/kg of total body weight. The daily dose for oral administration will preferably be from 0.1 to 300 mg/kg of total body weight. The daily dosage for administration by injection that includes intravenous, intramuscular, subcutaneous and parenteral injection as well as infusion techniques will preferably be from 0.1 to 300 mg/kg of total body weight. The daily vaginal dosage regime will preferably be from 0.1 to 300 mg/kg of total body weight. The daily topical dosage regimen will preferably be from 0.1 to 300 mg administered between one to four times daily. The transdermal concentration will preferably be that required to maintain a daily dose of from 1 to 300 mg/kg. For all the above-mentioned routes of administration, the preferred dosage is 0.1 to 300 mg/kg. The daily dosage regimen will preferably be from 0.1 to 300 mg/kg of total body weight.

The administered dosage of the Rho-kinase inhibitor may be modified depending on any superior or unexpected results which may be obtained as routinely determined with this invention.

Any of the routes and regiments of administration may be modified depending on any superior or unexpected results that may be obtained as routinely determined with this invention.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever

In the foregoing and in the following examples, all temperatures are set forth uncorrected in degrees Celsius and except where indicated, all parts and percentages are by weight/weight (w/w).

The entire disclosure of all applications, patents and publications cited herein are hereby incorporated by reference.

### EXAMPLES

### Example 1

### Inhibition by Fasudil of Abdominal Aortic Aneurysm and Cardiac Hypertrophy in Mice

ApoE-KO mice (6 month old) were infused with angiotensin II (1.44 mg/kg per day) for 30 days in the presence (n=18) or absence (n=20) of fasudil using methods described previously (Deng et al. (2003) Circ. Res. 92(5):510-7; Wang et al. (2001) Am. J. Pathol. 159(4):1455-64). A solution of fasudil was prepared by dissolving 1 mg/ml of fasudil in drinking water and provided to apoE-KO mice ad libitum. Daily water consumption was measured and the average daily dose of fasudil was calculated as approximately 130 mg/kg before any infusion of angiotensin II. Because angiotensin II increased daily water consumption, after 30 days of angiotensin II infusion, the average daily dose of fasudil was calculated as approximately 260 mg/kg. Both untreated angiotensin II-infused mice and angiotensin II-infused mice treated with fasudil consumed similar quantities of water during the course of this experiment. For some parameters measured, comparative data was also collected for C57 Black mice, the mouse strain from which the ApoE-KO mouse is derived.

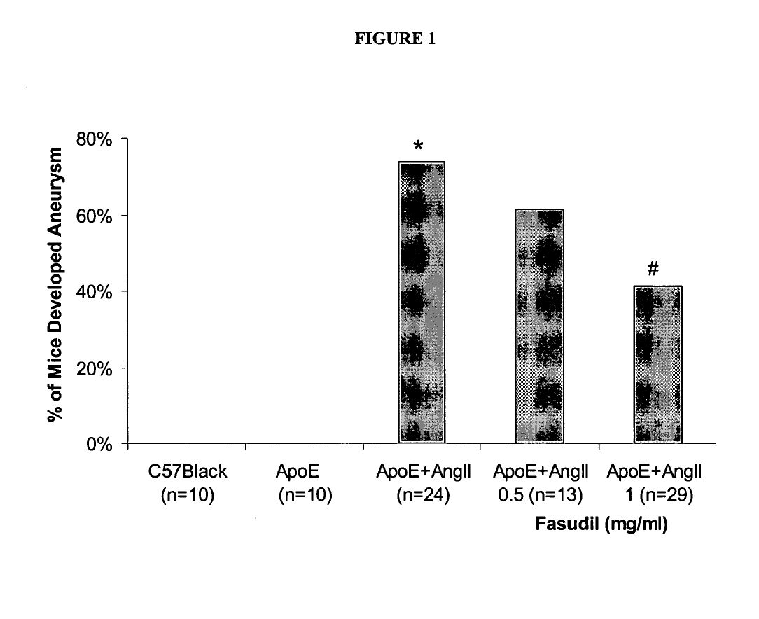

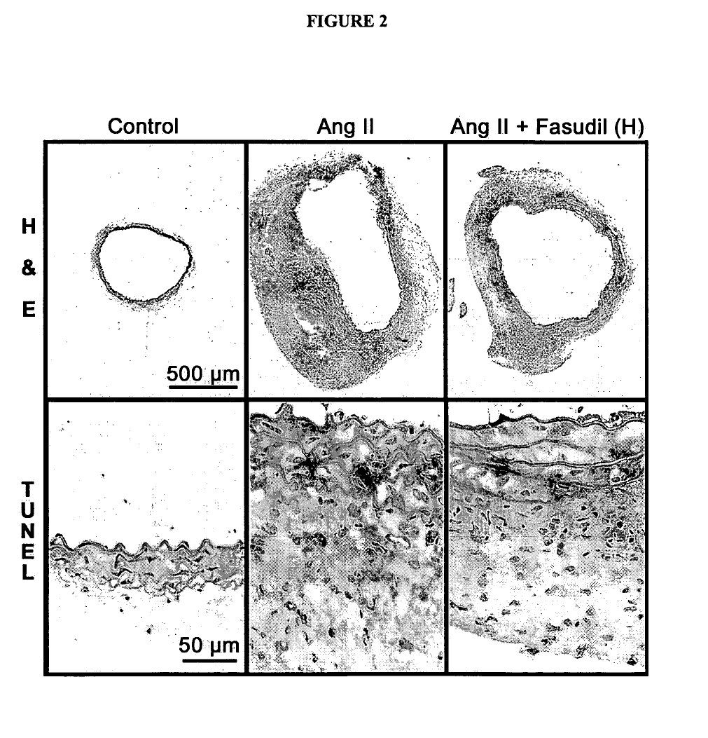

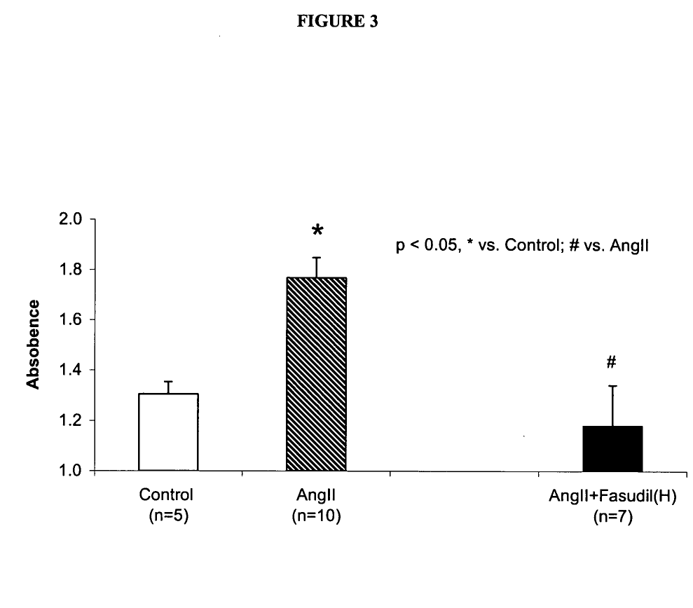

Infusion of angiotension II for 30 days in apoE-KO mice resulted in formation of abdominal aortic aneurysm in 72% of the ApoE-KO animals (n=18) (Table 1 & FIG. 1), as judged by postmortem direct measurement in agreement with previous work (Deng et al. (2003) Circ. Res. 92(5):510-7; Wang et al. (2001) Am. J. Pathol. 159(4):1455-64). In the supra-renal aorta from the ApoE-KO mice treated with Angiotensin II, strong TUNEL positive signals were detected in endothelium, media, as well as adventitia of the supra-renal aortas (FIG. 2), which was correlated to a significant increase in cytosolic DNA fragmentation (FIG. 3). In a second group of age matched apoE-KO mice (n=20) simultaneously infused with Angiotensin II as described above, oral administration of the Rho-kinase inhibitor, fasudil (HA-1077) (>100 mg/kg daily in drinking water) resulted in a decreased incidence of abdominal aortic aneurysm formation of 35%, a statistically significant decrease (p<0.05 vs. controls) (see Table 1). The reduction of aneurysm formation was associated with a decreased apoptosis (FIGS. 2 & 3). The plasma levels of the fasudil metabolite, hydroxyfasudil (HA-1100) were found to be 4.2±0.7 uM. Fasudil treatment had no effect on systolic blood pressure (SBP) or heart rate as measured by tail cuff instrumentation (Wang et al. (2001) Am. J. Pathol. 159:1455-64), no effect on serum cholesterol, or aortic arch atherosclerotic lesion area measured a described previously (Wang et al. ibid; Tham et al. (2002) Physiol. Genomics 11:21-30), and no effect on aortic stiffness as measured using methods described previously (Tham et al. (2002) Am. J. Physiol. Regul. Integr. Comp. Physiol. 283:R1442-9).

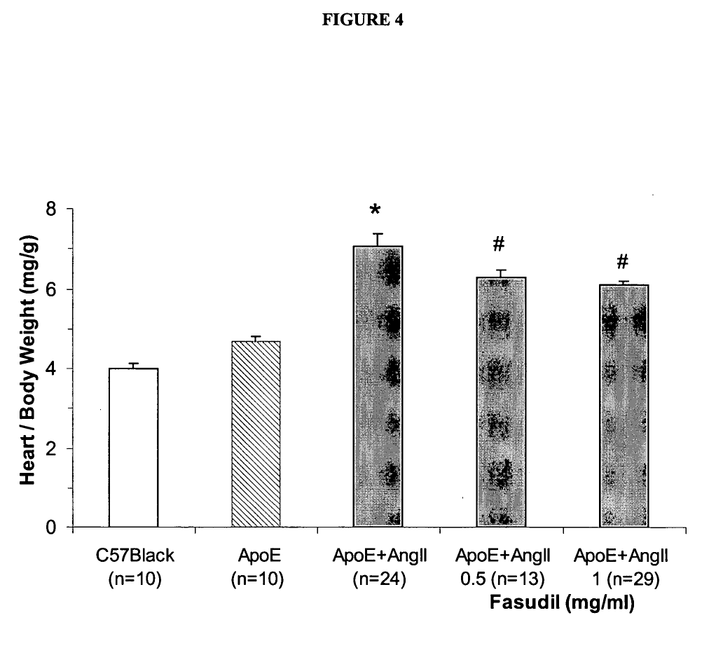

Angiotensin II treatment also caused cardiac hypertrophy, accompanied by up-regulation of gene expression of ANP and collagen III in the heart of apoE-KO mice. In order to determine whether fasudil treatment prevented cardiac hypertrophy in the same animals, the hearts were removed and wet weights measured then the heart tissue was prepared and examined. Treatment of apoE-KO mice with fasudil significantly reduced angiotensin-II induced cardiac hypertrophy (measured by heart weight or cardiomyocyte size) (Table 1 & FIG. 4). In addition, fasudil treatment reduced perivascular fibrosis, improved cardiac function, and normalized gene expression of ANP and collagen III in mice.

TABLE 1

| | Incidence | Diameter | Lesion | Heart/body | Myocyte |
|---|---|---|---|---|---|
| | (%) | (mm) | (%) | Wt | (mm²) |
| Control | 0 | 0.7 ± 0.0 | 5 ± 2 | 4.7 ± 0.1 | 253 ± 11 |
| Ang II | 74* | 2.1 ± 0.2* | 27 ± 2* | 7.1 ± 0.3* | 309 ± 12* |
| Ang II + | 41# | 1.5 ± 0.1# | 25 ± 3 | 6.1 ± 0.1# | 262 ± 16# |
| Fasudil | | | | | |

*p < 0.05, vs. Control, # p < 0.05, vs. Ang II

All publications and patents mentioned in the above specification are herein incorporated by reference. While the present invention has been described with reference to specific embodiments thereof, it will be understood by those skilled in the art that various changes may be made and equivalents substituted without departing from the true spirit and scope of the invention.

1. A method of treating aneurysm and cardiac hypertrophy, in a subject in need thereof, comprising administering a therapeutically effective amount of a rho-kinase inhibitor.

2. The method of claim 1, wherein the rho-kinase inhibitor is an isoquinoline derivative.

3. The method of claim 2, wherein the isoquinoline derviative is a compound of formula I 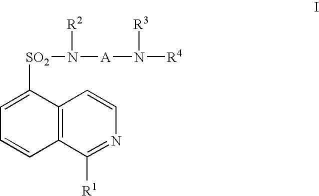
including salts and solvates thereof
wherein
R¹ is H, Cl, or OH; and
when R¹ is H,
A is an ethylene group optionally substituted with C₁₋₆ alkyl, phenyl or benzyl;
R² and R³ are directly bonded to each other thereby forming a trimethylene group optionally substituted with C₁₋₆ alkyl, phenyl or benzyl;
R⁴ is H or C₁₋₆ alkyl; and
when R¹ is Cl or OH
A is a C₂₋₆ alkylene group optionally substituted with C₁₋₆ alkyl;
R² and R³ are not bonded to each other and each is independently H or C₁₋₆ alkyl, or R² and R³ are directly bonded with each other thereby forming either an ethylene group optionally substituted with C₁₋₆ alkyl, or a trimethylene group optionally substituted with C₁₋₆ alkyl and
R⁴ is H, C₁₋₆ alkyl or amidino.

4. The method of claim 3 wherein the compound is fasudil or a salt or hydrate thereof.

5. The method of claim 3 wherein the compound is hydroxyfasudil or a salt or hydrate thereof.

6. The method of claim 1 wherein the rho-kinase inhibitor is the compound 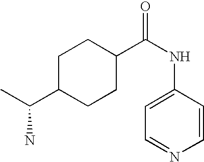
or a salt or hydrate thereof.

7. The method of claim 1 wherein the rho-kinase inhibitor is the compound 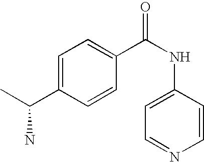
or a salt or hydrate thereof.

8. The method of claim 1 wherein the rho-kinase inhibitor is the compound 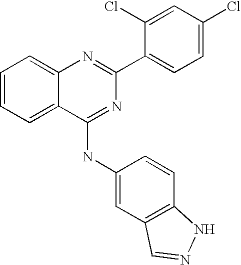
or a salt or hydrate thereof.

9. The method of claim 1 wherein the rho-kinase inhibitor is administered in combination with at least one other therapeutic agents selected from cholesterol lowering agents, antihypertensive agents, beta blocker drugs, calcium channel blockers, diuretics, nitrates, and ACE inhibitors.

10. A method of treating atherosclerosis or stenosis, in a subject in need thereof, comprising administering a therapeutically effective amount of a rho-kinase inhibitor.

11. The method of claim 10 wherein the rho-kinase inhibitor is an isoquinoline derivative.

12. The method of claim 11, wherein the isoquinoline derviative is a compound of formula I 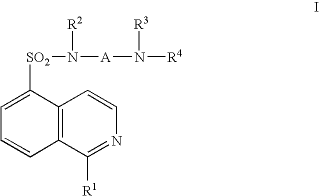
including salts and hydrates thereof
wherein
R¹ is H, Cl, or OH; and
when R¹ is H,
A is an ethylene group optionally substituted with C₁₋₆ alkyl, phenyl or benzyl;
R² and R³ are directly bonded to each other thereby forming a trimethylene group optionally substituted with C₁₋₆ alkyl phenyl or benzyl;
R⁴ is H or C₁₋₆ alkyl; and
when R¹ is Cl or OH
A is a C₂₋₆ alkylene group optionally substituted with C₁₋₆ alkyl;
R² and R³ are not bonded to each other and each is independently H or C₁₋₆ alkyl, or R² and R³ are directly bonded with each other thereby forming either an ethylene group optionally substituted with C₁₋₆ alkyl, or a trimethylene group optionally substituted with C₁₋₆ alkyl and
R⁴ is H, C₁₋₆ alkyl or amidino.

13. The method of claim 12 wherein the compound is fasudil.

14. The method of claim 13 wherein the compound is hydroxyfasudil.

15. The method of claim 10 wherein the rho-kinase inhibitor is the compound 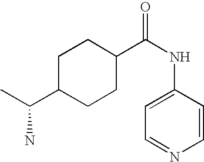
or a salt or hydrate thereof.

16. The method of claim 10 wherein the rho-kinase inhibitor is the compound 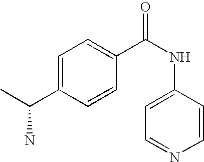
or a salt or hydrate thereof.

17. The method of claim 1 wherein the rho-kinase inhibitor is the compound 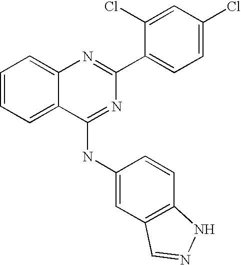
or a salt or hydrate thereof.
